(19) United States
(12) Patent Application Publication
(10) Pub. No.: US 2026/0240538 A1
(43) Pub. Date: Aug. 20, 2026

(54) Knotless Locking Loop Constructs and Methods of Tissue Repair

(71) Applicant: Arthrex, Inc., Naples, FL (US)

(72) Inventors: Jason A. VALENTIN, Fort Myers, FL (US); Nathanael I. GAMSO, Bonita Springs, FL (US); Shaun G. LEBLANC, Naples, FL (US)

(73) Assignee: Arthrex, Inc., Naples, FL (US)

(21) Appl. No.: 19/536,733

(22) Filed: Feb. 11, 2026

Related U.S. Application Data
(60) Provisional application No. 63/758,357, filed on Feb. 14, 2025.

(51) Int. Cl. A61B 17/04

(52) U.S. Cl. CPC A61B 17/0469; A61B 17/0401; A61B 17/0485; A61B 17/0487

(57) ABSTRACT

Tensionable, knotless, self-locking surgical constructs and methods for surgical repairs are disclosed. A surgical construct may be in the form of a repair suture with an attached shuttle/pull device to aid in formation of splices and tensionable, knotless loops.

### CROSS-REFERENCE TO RELATED APPLICATIONS

This application claims priority to U.S. Provisional Patent Application No. 63/758,357 filed Feb. 14, 2025, the disclosure of which is hereby incorporated by reference in its entirety herein.

### BACKGROUND

The disclosure relates to the field of surgery and, more specifically, to surgical loop constructs and associated methods of tissue repairs.

### SUMMARY

Surgical systems, assemblies, and methods for fixation of tissue are disclosed.

A surgical construct may create a knotless, self-locking, reinforced repair. A surgical construct may be made completely of suture to achieve fixation in bone without a separate anchoring body and with increased fixation. A surgical construct may enable tissue to tissue or tissue to bone fixation without the need for knots or complementary components. A surgical construct may create a knotless stitch around similar or different tissues.

A surgical construct may be a single suture that splices into itself so that it creates a loop with a static end and a tensionable end. The tensionable end enables the loop to be reduced until desired compression is achieved. The static end acts as an anchoring body ensuring that the construct maintains location during conversion and for the lifetime of the repair. The surgical construct does not require knot tying or additional components for fixating the suture construct after desired compression is achieved. The surgical construct may be employed in fixation of first tissue to second tissue, for example, fixation of soft tissue to soft tissue, or soft tissue to bone, or bone to bone.

Methods of tissue repairs are also disclosed. In some implementations, a knotless surgical construct may provide tissue fixation without any knot formation, by providing an all-suture repair that does not require a separate anchoring body or similar structure and which allows increased tissue fixation.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a knotless locking loop construct.

FIG. 2 illustrates another view of the loop construct of FIG. 1.

FIG. 3 illustrates a surgical repair with the construct of FIG. 2 at an initial stage.

FIG. 4 illustrates a surgical repair at a stage subsequent to that of FIG. 3.

FIG. 5 illustrates a surgical repair at a stage subsequent to that of FIG. 4.

FIG. 6 illustrates a surgical repair at a stage subsequent to that of FIG. 5.

FIG. 7 illustrates a surgical repair at a stage subsequent to that of FIG. 6.

FIG. 8 illustrates another surgical repair with the construct of FIG. 2.

FIG. 9 illustrates a surgical repair at a stage subsequent to that of FIG. 8.

FIG. 10 illustrates a surgical repair at a stage subsequent to that of FIG. 9.

FIG. 11 illustrates a surgical repair at a stage subsequent to that of FIG. 10.

FIG. 12 illustrates a surgical repair at a stage subsequent to that of FIG. 11.

FIG. 13 illustrates a surgical repair at a stage subsequent to that of FIG. 12.

FIG. 14 illustrates a surgical repair at a stage subsequent to that of FIG. 13.

FIG. 15 illustrates another surgical repair with the construct of FIG. 2.

FIG. 16 illustrates a surgical repair at a stage subsequent to that of FIG. 15.

FIG. 17 illustrates a surgical repair at a stage subsequent to that of FIG. 16.

FIG. 18 illustrates a surgical repair at a stage subsequent to that of FIG. 17.

FIG. 19 illustrates a surgical repair at a stage subsequent to that of FIG. 18.

FIG. 20 illustrates a surgical repair at a stage subsequent to that of FIG. 19.

FIG. 21 illustrates a surgical repair at a stage subsequent to that of FIG. 20.

FIG. 22 illustrates an exemplary fixation device of knotless locking loop construct of FIG. 1.

FIG. 23 illustrates another knotless locking loop construct.

FIG. 24 illustrates another knotless locking loop construct.

FIG. 25 illustrates another knotless locking loop construct.

FIG. 26 illustrates another knotless locking loop construct.

FIG. 27 illustrates another knotless locking loop construct.

FIG. 28 illustrates another knotless locking loop construct (before suture stuffing).

FIG. 29 illustrates another knotless locking loop construct (after suture stuffing).

FIGS. 30-33 illustrate subsequent steps of a method of assembling a knotless locking loop construct.

FIGS. 34-37 illustrate subsequent steps of another method of assembling a knotless locking loop construct.

FIG. 38 illustrates another knotless locking loop construct.

FIG. 39 illustrates another knotless locking loop construct.

FIG. 40 illustrates a surgical repair with the construct of FIG. 39 at an initial stage.

FIG. 41 illustrates a surgical repair at a stage subsequent to that of FIG. 40.

### DETAILED DESCRIPTION

A surgical construct may create a knotless, reinforced, self-locking repair.

A tissue repair system may include a tensionable surgical construct with a fixation device in the form of a flexible coupler (repair suture) and a shuttle suture (a shuttle/pull device) attached to the flexible coupler. The surgical construct may be a knotless construct. The surgical construct may be a self-locking construct. The flexible coupler may be a suture strand. The flexible coupler may be a suture strand provided with one or two tapered ends. The flexible coupler may be a tapered flexible braid with different widths and/or diameters on at least one end of the braid. A shuttle suture is attached (spliced) to the flexible coupler. A tissue repair system may be a soft tissue repair system.

The surgical construct may secure tissue (arthroscopic or endoscopic) with a flexible strand that does not require the surgeon to tie a knot. The construct pulls tissue to tissue together. The splice in the flexible strand increases the pull-out resistance of the construct and improves tissue cut-through resistance (by reducing the “cheese wire” effect) which is important in many clinical applications. The surgical construct may be employed in knotless fixation of first tissue to second tissue, for example, fixation of soft tissue to soft tissue, soft tissue to bone, or bone to bone.

Methods of knotless tissue repairs are also disclosed. In some implementations, a surgical construct may provide knotless first tissue to second tissue fixation, for example, knotless fixation of soft tissue (ligament, tendon, graft, etc.) to soft tissue without any knot formation and in a simple and fast manner.

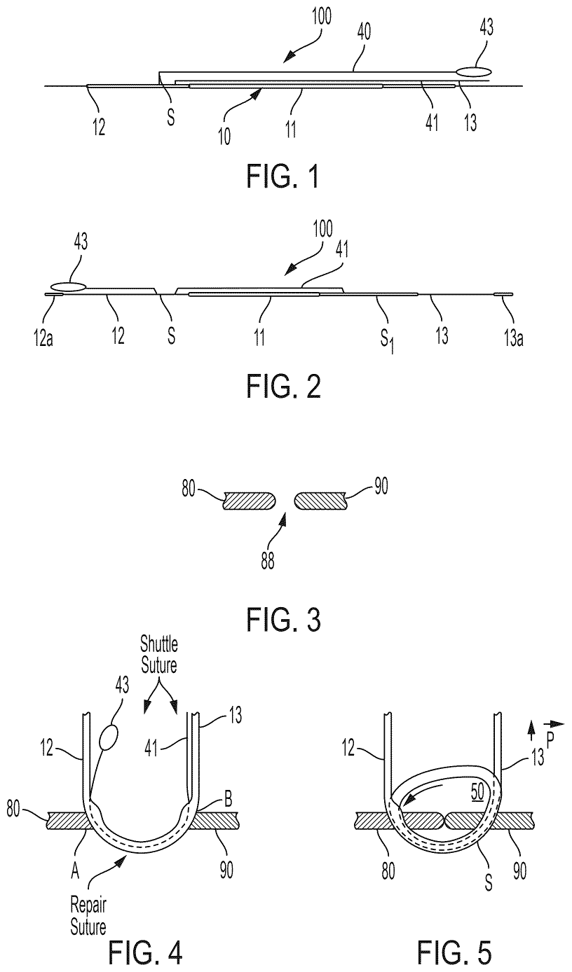

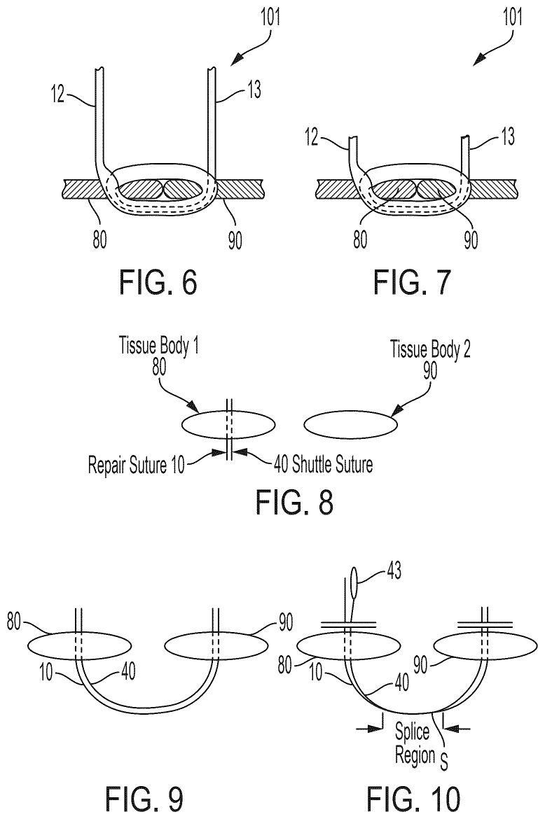

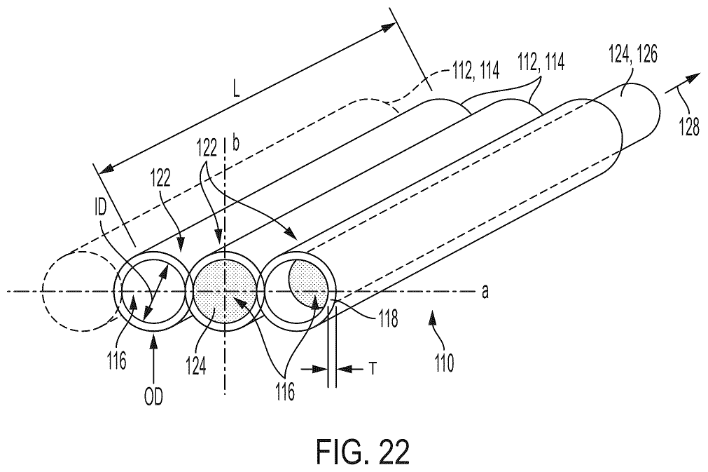

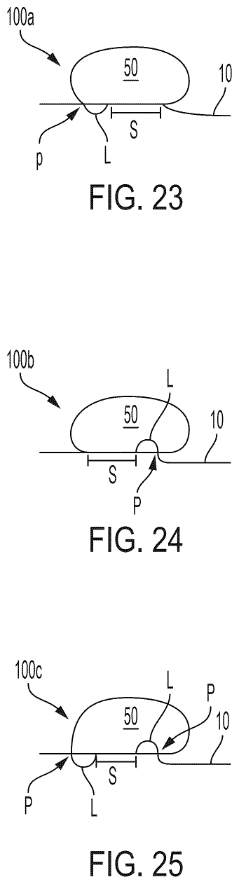

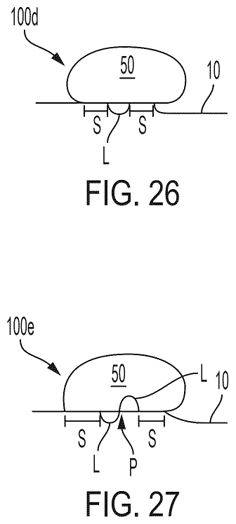

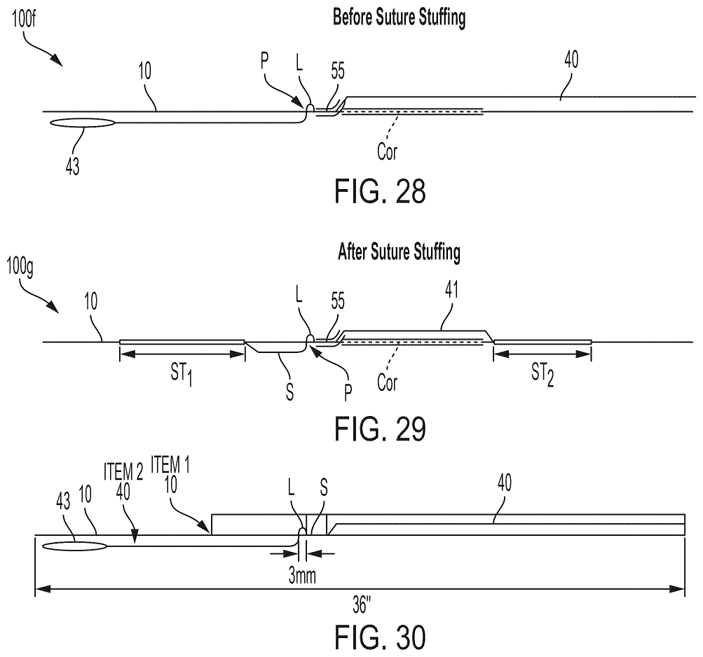

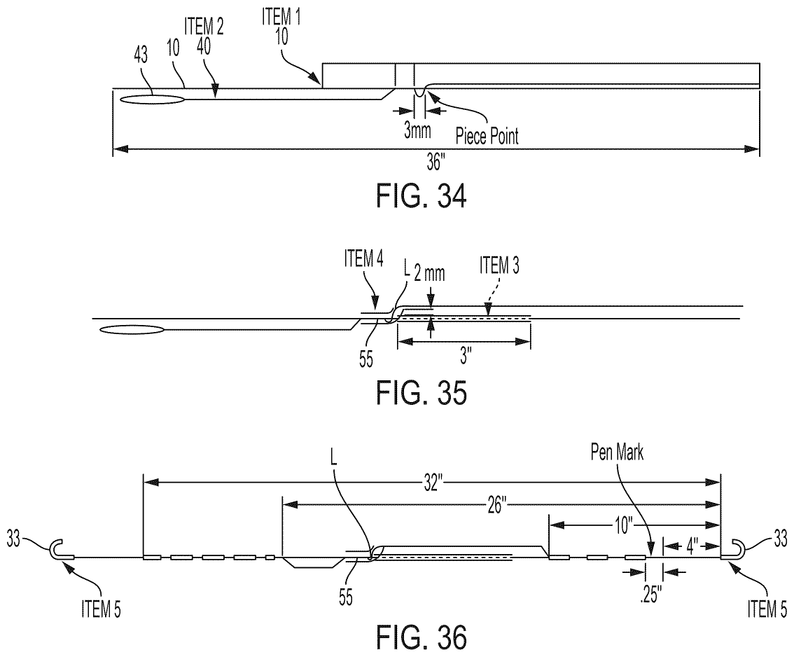

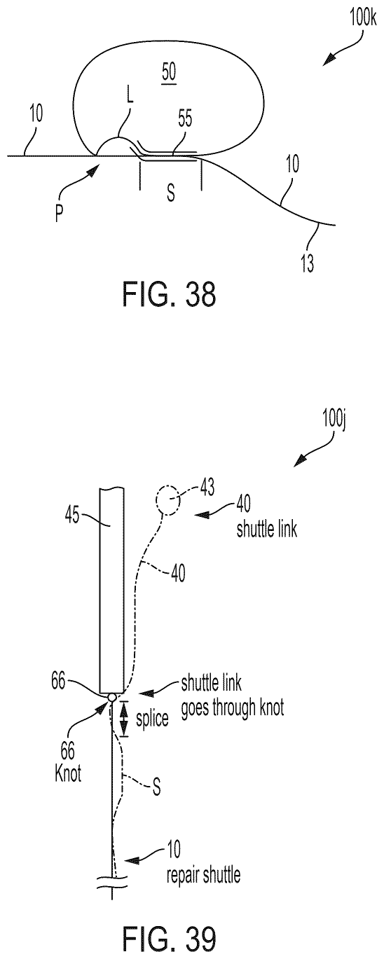

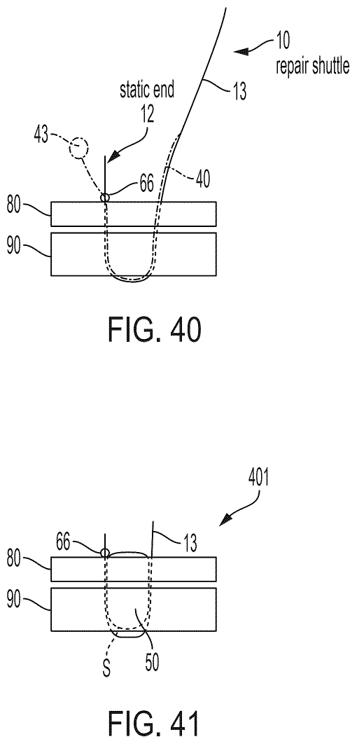

Referring now to the drawings, where like elements are designated by like reference numerals, FIGS. 1 and 2 illustrate surgical construct 100. FIGS. 3-7 illustrate an exemplary tissue repair 101 with surgical construct 100. FIGS. 8-14 illustrate another exemplary tissue repair 201 with surgical construct 100. FIGS. 15-21 illustrate yet another exemplary tissue repair 301 with surgical construct 100. FIG. 22 illustrates an exemplary fixation device 110 that may be employed in surgical construct of FIGS. 1 and 2. FIGS. 23-27 illustrate additional embodiments of knotless locking loop constructs 100a-100e of the present disclosure. FIGS. 28 and 29 illustrate knotless locking loop construct 100f (before suture stuffing) and 100g (after suture stuffing). FIGS. 30-33 illustrate subsequent steps of a method of assembling a knotless locking loop construct. FIGS. 34-37 illustrate subsequent steps of another method of assembling a knotless locking loop construct of the present disclosure. FIG. 38 illustrates surgical construct 100k. FIG. 39 illustrates surgical construct 100i. FIGS. 40 and 41 illustrate another exemplary tissue repair 401 with surgical construct 100i.

Surgical construct 100 may be a knotless locking loop construct, an implantable device, a surgical assembly, a surgical system, a tensionable, knotless construct, or a tensionable, knotless, self-locking, surgical anchor. Surgical construct 100 may be a single suture that splices into itself in such a way that creates a loop with a static end and a tensionable end. The tensionable end enables the loop to be reduced until desired compression is achieved.

Surgical construct 100 of FIGS. 1 and 2 may include a fixation device 10 and a shuttle/pull device 40. Fixation device 10 may be a flexible coupler 10 in the form of a suture; a suture tape; a braid; a repair suture; a round-flat suture; or a round-flat-round suture. In some implementations, fixation device 10 may be a round-flat-round suture tape 10 with tape 11 that may have a length of about 20 to about 40 inches (for example, about 36 inches), and ends 12, 13 that may have a length of about 1 to about 5 inches (for example, about 3 inches).

In some implementations, fixation device 10 may be provided as a one-piece machine taper construct in the form of a round to flat to round construct. The fixation device 10 may act as a soft anchor. In some implementations, fixation device 10 may be a simple round suture with ends formed also of round suture, having similar or different diameters. In some implementations, fixation device 10 may be a round suture with two tapered ends. In some implementations, fixation device 10 may be a round suture with a single tapered end. In some implementations, fixation device 10 may be a simple round suture having a constant diameter and no tapered ends.

FIGS. 1 and 2 illustrate an exemplary-only implementation wherein fixation device 10 includes a suture tape 11 terminating with tapered end 13 (having at least two different regions with different diameters) and tapered end 12. Ends 12, 13 may be tapered by splicing bifurcated, trifurcated, quadfurcated etc. ends through the unspliced ends and cutting the remaining ends. A shuttle/pull device 40 is attached to the fixation device 10. Shuttle/pull device 40 is attached to one of ends 12, 13 (for example, to end 12). End regions 12a, 13a may be glued to aid in limb passing.

Although, for simplicity, the implementations and embodiments below will be described with reference to flexible coupler 10 in the form of a round-flat-round suture, it must be understood that the disclosure is not limited to these exemplary-only implementations and embodiments, and encompasses any type of flexible coupler 10, as discussed above.

The flexible coupler 10 may be manufactured from any flexible material, for example, multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). Surgical constructs may be used with any type of flexible material or suture known in the art. The flexible coupler 10 may include elastic material. The flexible coupler 10 may consist essentially of elastic tapered suture.

In some implementations, flexible coupler 10 may be formed by cutting a braid off the spool and splitting the braid into a specific number of downsized sutures. Splitting the braid into downsized sutures may be conducted in any manner and for any number of times to form one or more tapered ends and help with the formation of the final construct.

In some implementations, flexible coupler 10 may be provided as a single strand of #5 or #2 machine taper suture. Flexible coupler 10 may be braided to a round suture with various diameters along its length. The diameter differentiation and furcation of suture may occur within the manufacturing process (e.g., braiding, weaving, sewing, stitching, etc.). In some implementations, flexible coupler 10 may be provided as a one-piece machine taper construct in the form of a round single suture strand of #5 or #2 machine taper suture. The flexible coupler 10 acts as a soft anchor.

FIGS. 1 and 2 also illustrate shuttle suture 40 (shuttle/pull device 40) extending through and attached to the flexible coupler 10. Shuttle/pull device 40 may be a suture passing instrument; a suture passer; a shuttle link; a FiberLink™ suture; or a Nitinol loop. Shuttle/pull device 40 is provided attached to flexible coupler 10, by being spliced, for example, to the flexible coupler 10 at splice region S (splice area S; simple splice S) inside the lumen of left end 12. The shuttle/pull device 40 may be any suture passing instrument or a shuttle link such as a FiberLink™ or a Nitinol loop provided with a loop/eye/eyelet. Shuttle/pull device 40 may include shuttle suture 41 and an eyelet/loop 43 for passing the flexible coupler 10.

In some implementations, and for ease of passage through tissue, end 41 of the shuttle/pull device 40 may be passed within the end 13 of the construct 100 (e.g., within the tensionable end 13 that engages splice S), as shown in FIG. 2. End 41 of shuttle/pull device 40 may be spliced through (inserted into) end 13 at splice S1 to form a single end or single passing limb for easier passage through first and second tissues 80, 90, as described below. The splice may be formed by piercing, joining or connecting strands or ends of strands or by interweaving the strands or ends of strands. A splice may extend between two or more splice points or pierce points (transverse openings). Interlacing yarns may include any weaving, braiding, and/or knitting.

Reference is now made to FIGS. 3-7. FIG. 3 illustrates a first tissue 80 to be attached or secured to a second tissue 90 with surgical construct 100. First tissue 80 may be soft tissue, hard tissue, connective tissue, bone, ligament, etc. Second tissue 90 may be soft tissue, hard tissue, connective tissue, bone, ligament, etc. Tissue gap 88 is located between first tissue 80 and second tissue 90.

Surgical construct 100 with shuttle/pull device 40 are deployed/passed through the first and second tissues 80, 90 as shown in FIG. 4. Deployment may be conducted with a suture passer, such as an Arthrex Scorpion™ suture passer, for tissue-to-tissue repair. In some implementations, end 41 of the shuttle/pull device 40 and tensionable end 13 are first passed through first tissue 80 at a location A and then are passed through second tissue 90 at a location B, as shown in FIG. 4.

Reference is now made to FIGS. 5-7. To form repair 101, free end 13 (together with end 41) may be passed through the eyelet/loop 43 of suture passing device 40. Suture passing device 40 is then pulled, thereby pulling free end 13 of the flexible coupler 10 towards the body of the flexible coupler 10, inside of the braid 10 (at splice S or splice region S) and then exiting the braid 10 to form a loop 50 and a stitch connecting first tissue 80 to second tissue 90. Loop 50 may be a flexible, closed, knotless, continuous loop. Loop 50 may be an adjustable and tensionable loop with an adjustable perimeter and length.

By pulling on free end 13 in the direction of arrow P (FIG. 5), the tensionable loop 50 shrinks and the tissue gap 88 becomes smaller to non-existent (reduced completely), tensioning the repair 101. Loop 50 connects first tissue 80 to second tissue 90 by passing through and around each tissue 80, 90 in a continuous, knotless manner. The suture ends 12, 13 of flexible coupler 10 may be tensioned and cut, as shown in FIG. 7. Alternatively, end 13 may be used for other tissue repairs that could include knotted or knotless steps, such as lateral tissue compression with accompanying interference anchors, for example. End 12 may be also cut or employed to incorporate one or more anchoring bodies, and as part of tissue repair 101.

Free end 12 of flexible coupler 10 is a static end. This end may incorporate an anchor body ensuring the construct maintains location during conversion and for the lifetime of the repair. Free end 13 of flexible coupler 10 is a tensionable end. Free end 13 of the flexible coupler 10 may be pulled to shrink the construct and the tensionable loop 50, and to compress the first tissue 80 to second tissue 90, providing final repair/construct 101 with increased compression of tissue.

For bone-to-bone repair, two through-holes (for example, two small-diameter holes) may be drilled into bone (for example, though different segments of the glenoid). The non-looped end 13 may be passed through a first hole and then through a second hole. The repair steps may be conducted as detailed above with respect to repair 101.

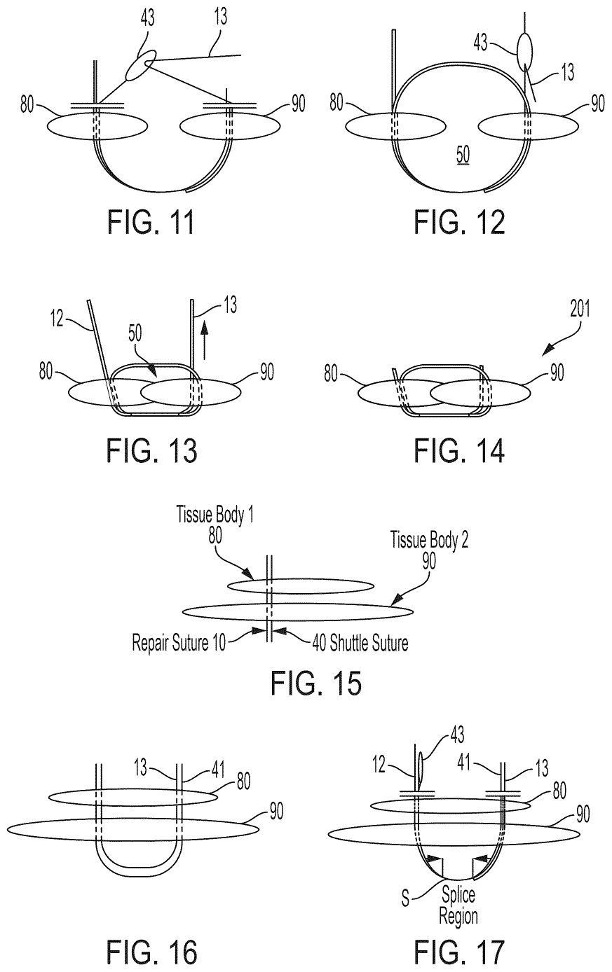

Reference is now made to FIGS. 8-14 which illustrate a side-to-side tissue repair 201:
FIG. 8: Tapered tail 13 of repair suture 10 and non-looped end 41 of the shuttle suture 40 are passed through one of the tissue body 80 from top to bottom.
FIG. 9: Tapered tail 13 of repair suture 10 and non-looped end 41 of the shuttle suture 40 are passed through the adjacent tissue body 90 from bottom to top.
FIG. 10: Pull on tapered tail 13 of repair suture 10 and non-looped end 41 of the shuttle suture 10 until the knotless mechanism splice region S is positioned below tissue repair region.
FIG. 11: Pass tapered tail 13 of repair suture 10 through the loop 43 of the shuttle suture 40.
FIG. 12: With the repair suture 10 captured by the shuttle suture loop 43, pull on the non-looped end 41 of the shuttle suture 40 resulting in the tapered end 13 of the repair suture 10 being pulled through the first tissue body 80, through the splice region S and through the second tissue body 90.
FIG. 13: Pull on tapered tail end 13 of the repair suture 10 until loop 50 is reduced and sufficient compression occurs between tissue group 1 (80) and tissue group 2 (90). Cut away tapered tail 13 and static end 12 of repair suture 10 close to tissue 80, 90.
FIG. 14 illustrates repair 201.

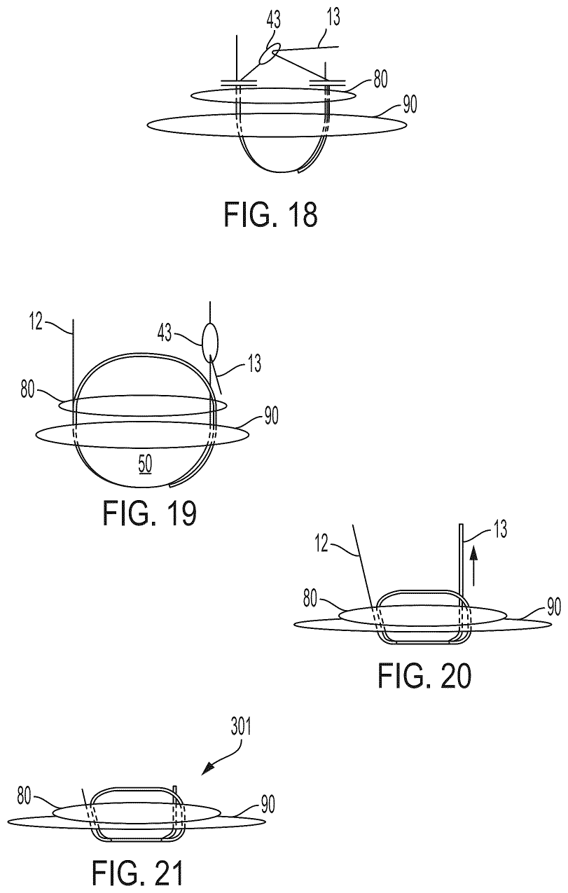

Reference is now made to FIGS. 15-21 which illustrate an over-under tissue repair 301:
FIG. 15: Tapered tail 13 of repair suture 10 and non-looped end 41 of the shuttle suture 40 are passed through top tissue body 1 (80) and tissue body 2 (90) from top to bottom.
FIG. 16: Tapered tail 13 of repair suture 10 and non-looped end 41 of the shuttle suture 40 are passed tissue body 2 (90) and tissue body 1 (80) from bottom to top.
FIG. 17: Pull on tapered tail 13 of repair suture 10 and non-looped end 41 of the shuttle suture 40 until the knotless mechanism splice region S is positioned below tissue repair region.
FIG. 18: Pass tapered tail 13 of repair suture 10 through the loop 43 of the shuttle suture 40.
FIG. 19: With the repair suture 10 captured by the shuttle suture loop 44, pull on the non-looped end 41 of the shuttle suture 40 resulting in the tapered end 13 of the repair suture 10 being pulled through the first tissue body 80, through the splice region S and through the second tissue body 90.
FIG. 20: Pull on tapered tail end 13 of the repair suture 10 until loop 50 is reduced and sufficient compression occurs between tissue group 1 (80) and tissue group 2 (90). Cut away tapered tail 13 and static end 12 of repair suture 10 close to tissue 80, 90.
FIG. 21 illustrates repair 301.

Although the implementations above have been described with reference to splice region S being situated at about a mid-location between first and second tissues 80, 90 (as shown in FIGS. 10 and 17, for example), it must be understood that the disclosure is not limited to these exemplary-only implementations and contemplates embodiments wherein the splice region is maintained anywhere in between the first and second tissues 80, 90. Thus, the location and formation of the splice are not essential as long as the knotless construct is formed. Accordingly, the splice can be also formed in an “in-an-out” manner or in any other manner known in the art, and as long as it allows the formation of at least one knotless adjustable loop around tissues.

In addition, although the implementations above have been described with reference to repair suture 10 and shuttle link 40 passing through tissue 80 (tissue body 1) and tissue 90 (tissue body 2) at adjacent yet spaced-apart locations (as shown in FIGS. 8-21), it must be understood that the present disclosure is not limited to these exemplary-only implementations. Thus, the disclosure also contemplates embodiments wherein repair suture 10 and shuttle link 40 pass through at least one of tissue 80 (tissue body 1) and tissue 90 (tissue body 2) at the same location, for example, having similar entry and exit points through tissues 80, 90. The distance between the entry points of repair suture 10 and shuttle link 40 through either of tissue 80 or 90 can be between 0 millimeters to about few millimeters. In certain implementations, the distance between the entry points of repair suture 10 and shuttle link 40 through either of tissue 80 or 90 can be of about few inches, depending on the specific surgical repair and physical characteristics of tissues 80, 90 to be stitched/attached. Similarly, the distance between the exit points of repair suture 10 and shuttle link 40 through either of tissue 80 or 90 can be between 0 millimeters to about few millimeters, or of about few inches, depending on the specific surgical repair and physical characteristics of tissues 80, 90 to be stitched/attached. In yet additional implementations, the lines connecting entry and exit points of repair suture 10 and shuttle link 40 through either of tissue 80 or 90 can be non-parallel, for example, when repair suture 10 and shuttle link 40 enter tissue 80 at a same entry point yet exit tissue 80 at different exit points, or vice-versa.

Reference is now made to FIG. 22 which illustrates an exemplary construct 110 that may be employed with the above-described surgical constructs and related methods of tissue repair. The disclosure may be implemented to include various textile constructs 110 that may provide for one or more interconnected features 112. The interconnected features 112 may comprise a plurality of tubular structures 114 that may form adjacent lumens 116 extending along a length L. In the example shown, three adjacent tubular structures 114 are interlinked, such that the neighboring lumens 116 are connected by and/or share walls 118. Intertwined or braided yarns or strands may form the walls 118 to a thickness T extending between an outside diameter OD and an inside diameter ID. The thickness T of the walls 118 and the corresponding diameters OD, ID may vary based on the strand count, interlinked configuration, or strand thickness of the strands forming the textile construct 110. Though described as tubular shapes having an outside diameter OD and an inside diameter ID, the cross-section of the tubular structures 114 may be elliptical, rectangular, or form various profile shapes along the length L that may intersect along one or more adjacent sections of the walls 118.

In various implementations, the tubular structures 114 may be formed by one or more layers of interlinked strands manufactured through various techniques. In a braided configuration, the tubular structures 114 may be formed by maneuvering the corresponding strands through complex, interlinked paths along which bobbin carriers dispense the strands in coordination. In this way, the proportions, thickness, and/or materials associated with the tubular structures 114 and other interconnected features 112 may vary along the length L. As shown in FIG. 22, the strands or yarns forming the tubular structures 114 may travel along interlinked paths and form an extended figure “8” from a series of interlinked loops 122. For example, the interlinked loops 122 may be formed by three or more of the strands or yarns traversing intertwined paths at an angle of approximately 45° or greater. In various implementations, three, four, or more carriers may traverse preconfigured, intersecting paths causing the corresponding strands or yarns to be interlaced defining the profile shape of the walls 118. In this way, the tubular structures 114 may be formed by the interlaced yarns or strands as defined by the corresponding paths of the carriers traversing the profile shape of the walls 118.

The shape of the interconnected features 112, exemplified as the tubular structures 114, may be adjusted along the length L of the textile construct 110 by changing the paths of the carriers among a variety of tubular shapes (e.g., round or rectangular tubes) or closed/filled cross-sections to suit various applications. The interlinked loops 122 extend along the length L and may define a major axis (a) extending perpendicular to a minor axis (b). The textile construct 110 may resist bending along the major axis (a) due to the alignment of two or more associated tubular structures 114 or interconnected features 112. Comparatively, the textile construct 110 may be more flexible along the minor axis (b). Accordingly, the textile construct 110 may provide directional flexibility or rigidity along one or more axes (a, b) defined by the alignment of two or more of the tubular structures 114.

Still referring to FIG. 22, a core 124 may be disposed in one or more of the lumens 116. In various implementations, the lumen 116 may provide for a passage or pocket that may extend through a portion of the construct 110. In such cases, the lumen 116 may pass or house various surgical implements (e.g., sutures, needles, etc.) for storage, organization, or management along all or part of the length L. The core 124 may be interlinked or affixed with the tubular wall 118 or correspond to a removable insert 126 that may closely fit within the tubular wall 118. Accordingly, the core 124 or insert 126 may be implemented to permanently or selectively adjust the stiffness or shape of the textile construct 110 along the length L. An example of the removal of the insert 126 is illustrated by an arrow 128. Though not illustrated in FIG. 22, one or more of the interconnected features 112 presented as tubular structures 114 may be formed as flat or collapsed sections over a portion or the entirety of the length L. The flat section(s) may be formed from the strands or yarns of the tubular walls 118 braided together or interconnected over transition sections along the length L to form a tape-like or ribbon-like structure.

The materials forming the textile construct 110 may correspond to continuous or semi-continuous yarns, strands, or filaments. Depending on the application, the strands or filaments may be formed from natural or synthetic materials. Various materials may also be implemented for the cores 124 or inserts 126. For example, one or more strands of wire, thread, and/or fabric may be included in one or more of the lumens 116. In some cases, a solid or semi-rigid core may be enclosed within one or more tubular structures 114, which may correspond to polymeric or metallic inserts. In some implementations, padding or stuffing may be enclosed within one or more tubular structures 114. In various implementations, the cores 124 or inserts 126 may limit or control the flexibility along the length L and may also serve as a cushion or fill that may increase or maintain volumetric proportions of the corresponding tubular structure 116 when deployed. The cores 124 or inserts 126 may be incorporated as one or more preformed shapes or bendable materials that may maintain a desired shape (e.g., malleable steel, memory alloys, etc.). The cores 124 or inserts 126 may also include a combination of multiple materials (e.g., a padding layer, a stiffening layer, stiffening strands within a malleable core, etc.). Accordingly, the textile construct 110 may be constructed to provide a variety of functional characteristics to suit a wide range of applications.

Reference is now made to FIGS. 23-37, which illustrate additional implementations of the knotless locking loops and methods of assembly of the present disclosure.

FIGS. 23-27 depict surgical constructs 100a, 100b, 100c, 100d, 100e in the form of various knotless mechanisms having a tortuous path. In an implementation, a tortuous path can include a pierce point and/or a splice and/or a loop. In an implementation, a tortuous path can include a plurality of pierce points and/or a plurality of splices and/or a plurality of loops, by themselves or in any combination. Surgical constructs 100a, 100b, 100c, 100d, 100e include a tortuous path in the form of an added pierce point P that can be provided prior or after the splice S. Surgical constructs 100a, 100b, 100c, 100d, 100e can include one or more additional gaps or loops L provided between the pierce point P and splice S.

To form exemplary pierce point P, loop L and splice S, suture 10 which is a typical coreless suture with a suture lumen (i) is passed through itself by breaking both walls of the suture lumen from a lower wall/side to an upper wall/side (for example) and exits the suture lumen to form pierce point P and then (ii) reenters the suture lumen by breaking only one wall of the suture lumen (for example, the upper wall of the suture lumen) to form loop L and then (iii) extends within the suture lumen along a longitudinal axis of the suture lumen and for a defined distance to form splice S.

Using a combination of splices, gaps or loops, and pierce points creates a torturous path for suture 10 to travel, resulting in increased inhibition of slippage after tightening. Pierce points and gaps can occur before, after, or anywhere within the splice location any number of times needed to acquire the required strength. The addition of splices, pierce points and/or gaps/loops improves suture management and augments the overall knotless fixation. Knotless suture construct 100a, 100b, 100c, 100d, 100e can include a pierce point that forms a tiny loop (gap) and then a splice, or a splice followed by a pierce point, and can have the tails tucked in the suture.

Surgical construct 100a, 100b, 100c, 100d, 100e may be a knotless locking loop, an implantable device, a surgical assembly, a surgical system, a tensionable, knotless construct, or a tensionable, knotless, self-locking, surgical anchor. Surgical construct 100a, 100b, 100c, 100d, 100e may be a single suture that splices into itself in such a way that creates a loop with a static end and a tensionable end. The tensionable end enables the loop to be reduced until desired compression is achieved.

Reference is now made to FIGS. 28 and 29 which illustrate surgical constructs 100f and 100g provided without and with suture stuffing, respectively. FIG. 28 illustrates surgical construct 100f with repair suture 10 and shuttle suture 40 before suture stuffing. FIG. 29 illustrates surgical construct 100g with repair suture 10 and shuttle suture 40 after the suture stuffing. For minimizing the number of tails in any given construct, suture can be unionized to form one tail, permanently or temporarily. One advantage is that it limits the number of passes required for a repair, as the surgeon does not need to group the suture limbs/tails together for easy passing. Another advantage is that it aids in suture management.

FIG. 29 illustrates construct 100g after suture stuffing. Both suture ends of shuttle suture 40 are brought together with suture 10 to form stuffed suture region ST1, ST2 provided on opposite sides of splice S, pierce point P, loop L and cored region Cor. Surgical constructs 100f, 100g of FIGS. 28 and 29 also include a sheath 55 provided adjacent pierce point P and loop L. Sheath 55 can be an exemplary 10 mm sheath for CT or similar device.

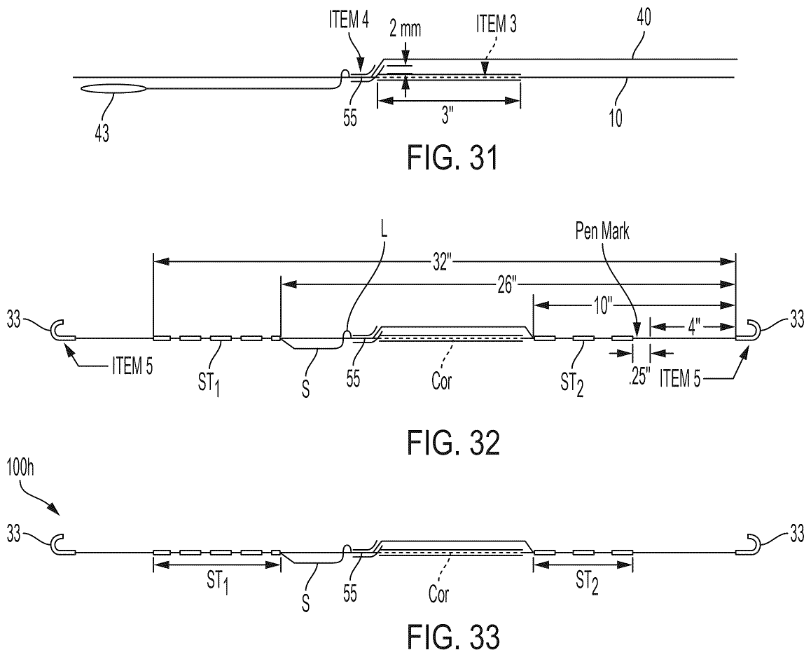

Final construct 100k (FIG. 38) illustrates construct 100f of FIG. 28 after removal of shuttle link 40 and passage of repair suture 10 to form tensionable loop 50. As detailed above, tapered tail 13 of repair suture 10 is passed through loop 43 of the shuttle suture 40; the repair suture 10 is captured by the shuttle suture loop 43; the non-looped end 41 of the shuttle suture 40 is pulled resulting in the tapered end 13 of the repair suture 10 being pulled through splice region S and sheath 55, forming tensionable loop 50 around and/or through first and second tissues 80, 90. In all the embodiments detailed above, the distance between pierce point P and beginning of splice S does not change and it remains constant. The pierce point P and the sheath 55 do not interact with the suture 10. The distance between pierce point P and starting of splice S remains constant even when tensioning of the repair takes place with the in and out knotless mechanism detailed above. The pierce point P, loop L, and splice S are formed as detailed above with respect to FIGS. 23-27.
FIGS. 30-33 illustrate exemplary steps of assembling knotless locking loop construct 100h (FIG. 33) which is about similar to construct 100g of FIG. 29 but differs in that construct 100h has needles 33 provided on each end of suture ends 12, 13. Construct 100h is a CT knotless stitch.
FIG. 30: Pierce and splice Item 2 (shuttle suture 40) into Item 1 (repair suture 10) as shown to form splice S and loop L.
FIG. 31: Splice Item 3 (stuffing construct) into Item 1 as shown. Ensure Item 3 is fully encapsulated to form cored region Cor (adjacent decored regions). Pass Item 2 completely through Item 4 (a sheath 55 such as a 10 mm sheath for CT). Pass Item 1 through sheath 55 and pierce through sheath 55 (for example, at about 2 mm from end).
FIG. 32: Splice looped and non-looped ends of Item 2 into Item 1 to form regions ST1 and ST2.
FIG. 33: After both ends are spliced and prior to cutting, pull non-looped end of Item 2 to remove all slack. Ensure Item 2 ends are fully encapsulated after cutting. Affix Item 5 (needle 33) to both ends of the Item 1 to form construct 100h. Mark Item 1 where indicated.

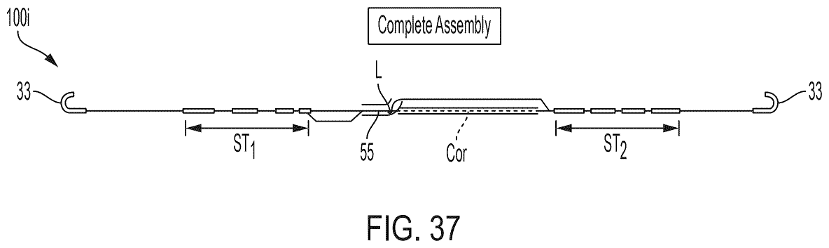

FIGS. 34-37 illustrate subsequent steps of assembling surgical construct 100i (FIG. 37) which is about similar to construct 100h (FIG. 33) but differs in that loop L is located within Item 4 (sheath 55) and not outside of it. Construct 100i is a CT knotless stitch.

FIG. 34: Pierce and Splice Item 2 into Item 1 as shown to the specified dimensions.

FIG. 35: Splice Item 3 into Item 1 as shown. Ensure Item 3 is fully encapsulated. Pass Item 2 completely through Item 4 (sheath 55). Pass Item 1 through sheath 55 and pierce through sheath 55 (about 2 mm from end).

FIG. 36: Splice looped and non-looped ends of Item 2 into Item 1, as shown. After both ends are spliced and prior to cutting, pull non-looped end of item 2 to remove all slack. Ensure Item 2 ends are fully encapsulated after cutting. Affix Item 5 to both ends of the Item 1. Mark Item 1 where indicated with marker.

Surgical construct 100h (FIGS. 33) and 100i (FIG. 37) can be passed through and/or around tissue 80, 90 (as shown, for example, in FIGS. 8-14) and needles 33 cut to allow formation of tensionable loop 50, loop (gap) L and splice S as part of a final repair and construct (such as that of FIG. 21 or 23, for example). As detailed above, the distance between pierce point P and beginning of splice S does not change and it remains constant while tensioning of the repair takes place and as part of the final repair.

Reference is now made to FIGS. 39-41, which illustrate additional implementations of the knotless locking loops and methods of assembly of the present disclosure. Construct 100j of FIG. 39 is about similar to the constructs described above in that it also includes repair suture 10 and shuttle link 40 (shuttle/pull device 40) loaded onto it; however, construct 100j also includes an exemplary positioning device 45 (for example, straw 45) and an additional reinforcement or reinforcing structure 66. In an implementation, the reinforcing structure 66 may include at least one knot 66. In an implementation, the reinforcing structure may include two or more knots 66. The plurality of knots 66 may be provided adjacent to each other and/or in contact with each other to provide additional strength and a more tortuous path. Knot(s) 66 can be provided prior to the loops to provide additional strength.

As shown in FIG. 39, shuttle link 40 passes through exemplary knot 66 and into the repair suture 10 so that conversion is achieved through knot 66 and splice S. Knot 66 adds additional strength to the overall repair. During conversion and formation of tensionable, adjustable loop 50 of final repair 401 (FIG. 40), repair suture 10 does not pass through knot 66; knot 66 rests over and/or in contact with one of tissues 80, 90 in the final repair.

FIGS. 40 and 41 depict construct 100j with tensionable limb 13 of repair suture 10 passed through tissue 80 and tissue 90 at different locations and tissue entry points as shown in FIG. 40. Knot 66 and loop 43 of shuttle link 40 remain over and on top of tissues 80, 90 to allow formation of tensionable loop 50 (FIG. 41), once end 13 has been passed through loop 43 and shuttle link 40 has been pulled out of the construct 100j.

Although the addition of positioning device 45 and reinforcement structure 66 has been described only with respect to exemplary surgical construct 100j, it must be understood that reinforcement structure 66 (one or more knots 66) can be added to any of the previously-described surgical constructs 100,100a, 100b, 100c, 100d, 100e, 100f, 100g, 100h, 100i, 100k. The addition of one or more knots 66 can be accompanied with or without the positioning device 45, according to the surgeon's preference.

The construct secures tissue (arthroscopic or endoscopic) with a suture that does not require the surgeon to tie a knot. The construct pulls tissues together. The construct may include a flexible coupler that is not tapered. The construct may include a flexible coupler that is tapered/furcated. The furcation(s) in the flexible coupler allow the construct to improve tissue cut-through resistance (by reducing the “cheese wire” effect) which is important in many clinical applications. The construct may be employed in knotless fixation of first tissue to second tissue, for example, fixation of soft tissue to soft tissue, or soft tissue to bone, or bone to bone.

The constructs, systems, and assemblies of the present disclosure may be employed in numerous knotless or knotted soft tissue repairs and fixations, for example, fixation of soft tissue to soft tissue. The device is a single suture that splices into itself in such a way that creates a loop with a static end and a tensionable end. The tensionable end enables the loop to be reduced until desired compression is achieved. The static end can incorporate an anchoring body ensuring the construct maintains location during conversion and after the lifetime of the repair. The device does not require knot tying or additional components for fixating the suture construct after desired compression is achieved. The construct enables tissue to tissue or tissue to bone fixation without the need for knots or complimentary components.

A surgical construct 100,100a, 100b, 100c, 100d, 100e, 100f, 100g, 100h, 100i, 100j, 100k may comprise a flexible coupler 10 with two opposing ends 12, 13; and a shuttle/pull device 40 attached to the flexible coupler 10. Ends 12, 13 may be tapered ends and may have similar or different diameters/widths. At least one of ends 12, 13 may be tapered and may have a diameter smaller than the diameter of flexible coupler 10. The surgical construct 100,100a, 100b, 100c, 100d, 100e, 100f, 100g, 100h, 100i, 100j, 100k may be a knotless locking loop construct. The flexible coupler 10 may be a braid or round suture. The ends 12, 13 may form at least one splice within the flexible coupler 10 and at least one loop 50 around and through a first tissue 80 to be secured to a second tissue 90. Loop 50 may be an adjustable, knotless, closed, continuous loop.

Methods of soft tissue repair 101 which do not require tying of knots and allow adjustment of both the tension of the suture and the location of first tissue with respect to second tissue, while providing self-locking mechanism, are disclosed.

A method of knotless tissue repair 101, 201, 301, 401 may comprise inter alia the steps of: (i) positioning a loop construct 100,100a, 100b, 100c, 100d, 100e, 100f, 100g, 100h, 100i, 100j, 100k at a first location A of a first tissue 80 and at a second location B of a second tissue 90, the loop construct 100 comprising a flexible coupler 10 preloaded with a shuttle/pull device 40 (shuttle link 40) and having a first end 12 and a second end 13; and (ii) passing one of the first and second ends 12, 13 through the flexible coupler 10 by employing the shuttle/pull device 40, to form a loop 50 around and through each of the first tissue 80 and the second tissue 90. Loop construct 100,100a, 100b, 100c, 100d, 100e, 100f, 100g, 100h, 100i, 100j, 100k may be a knotless locking loop construct. Loop 50 may be an adjustable, knotless, self-locking, closed, continuous loop. The method may further include (iii) pulling on the one of the first and second ends 12, 13 to tension the repair 101; decrease the dimensions of the loop 50; and decrease a distance 88 between the first tissue 80 and the second tissue 90. The method may further include (vi) cutting off the first and second ends 12, 13 or employing at least one of the first and second ends 12, 13 in additional surgical steps. Step (i) may be conducted by (a) passing one of the first and second ends 12, 13 (e.g., end 13 that is not spliced with the shuttle/pull device 40) through the first tissue 80, from a first surface to a second opposite surface of the first tissue 80; and (b) passing the one of the first and second ends 12, 13 (e.g., end 13 that is not spliced with the shuttle/pull device 40) through the second tissue 90, from a first surface to a second opposite surface of the second tissue 90, so that the loop 43 of the shuttle/pull device 40 extends above the first surface of the first tissue 80. Loop 50 may have an adjustable perimeter and length. The shuttle/pull device 40 may be a shuttle suture with a looped end 43 and a non-looped end 41. Repair 101, 201, 301, 401 may be a knotless stitch. Repair 101, 201, 301, 401 may be a soft tissue repair. Repair 101, 201, 301, 401 may be a rotator cuff repair.

Flexible coupler 10, 110 and ends 12, 13 may include any flexible material, for example, multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). FiberWire® suture is formed of an advanced, high-strength fiber material, namely ultrahigh molecular weight polyethylene (UHMWPE), sold under the tradenames Spectra (Honeywell) and Dyneema (DSM) fibers, braided with at least one other fiber, natural or synthetic, to form lengths of suture material.

Flexible coupler 10, 110 and ends 12, 13 may be also formed of a stiff material, or combination of stiff and flexible materials, particularly for the regions of the coupler that are passed/spliced through the body of the coupler and depending on whether they are employed with additional fixation devices. In addition, flexible coupler 10, 110 and ends 12, 13 may be also coated and/or provided in different colors for easy manipulation during the surgical procedure. Flexible coupler 10, 110 and ends 12, 13 may be also provided with one or more suture passers such as needles 33. The knotless constructs and self-locking soft anchors of the present disclosure may be used with any type of flexible material or suture that may be weaved or passed through itself.

Various structural elements of surgical construct 100, 100a, 100b, 100c, 100d, 100e, 100f, 100g, 100h, 100i, 100j, 100k may be visually coded, making identification and handling of the sheath and suture legs simpler. Easy identification of suture in situ is advantageous in surgical procedures, particularly during arthroscopic surgeries, endoscopic and laparoscopic procedures.

The surgical constructs of the present disclosure may be employed in endoscopic surgery. The term “endoscopic surgery” refers to surgical procedures within a patient's body through small openings as opposed to conventional open surgery through large incisions. Additionally, surgical constructs as disclosed herein may be utilized in other general surgical and specialty procedures such as soft tissue repairs.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A knotless locking loop repair, comprising:
a repair suture; and
a shuttle suture attached to the repair suture;
wherein the repair suture and the shuttle suture pass through a first tissue and through a second tissue, and
wherein the repair suture and the shuttle suture are configured to form at least one loop around and through each of the first tissue and the second tissue, and to bring together the first tissue and the second tissue.

2. The knotless locking loop repair of claim 1, wherein the at least one loop is a knotless, tensionable loop.

3. The knotless locking loop repair of claim 1, wherein the repair suture is a suture braid with a first end on one side of the suture braid, and a second end on another side of the suture braid, and wherein at least one of the first end and the second end is a tapered end.

4. The knotless locking loop repair of claim 3, wherein pulling on one of the first and second ends of the suture braid reduces the at least one loop and tensions the repair.

5. The knotless locking loop repair of claim 1, wherein the repair suture consists essentially of elastic suture.

6. The knotless locking loop repair of claim 1, wherein the first tissue is soft tissue, hard tissue, or connective tissue.

7. The knotless locking loop repair of claim 1, wherein the second tissue is soft tissue, hard tissue, or connective tissue.

8. A method of knotless tissue repair comprising:
passing through a first tissue a locking loop construct consisting of a braid terminating into a first tapered end and a second tapered end, and a shuttle/pull device attached to one of the first and second tapered ends, wherein the first tapered end is opposite the second tapered end;
passing the locking loop construct through a second tissue to be attached to the first tissue; and
passing the other one of the first and second tapered ends through an eyelet of the shuttle/pull device and pulling the shuttle/pull device out of the braid to form a loop around the first tissue and the second tissue.

9. The method of claim 8, wherein the loop is a knotless, tensionable loop.

10. The method of claim 9, further comprising:
reducing the knotless, tensionable loop by pulling on the other one of the first and second tapered ends; and
cutting at least one of the first and second tapered ends.

11. The method of claim 8, wherein passing the locking loop construct through a second tissue to be attached to the first tissue is conducted while the locking loop construct remains within the first tissue.

12. The method of claim 8, wherein the first tissue is bone, tendon, or ligament and the second tissue is bone, tendon, or ligament.

13. The method of claim 8, wherein the locking loop construct consists essentially of elastic suture.

14. A knotless locking stitch repair, comprising:
a repair suture passed through a first tissue at a first location and through a second tissue at a second location,
wherein the repair suture passes from a top surface to a bottom surface of the first tissue, and from a bottom surface to a top surface of the second tissue, and then through itself to form a splice and a knotless, tensionable loop around and through each of the first tissue and the second tissue, and to bring together the first tissue and the second tissue.

15. The knotless locking stitch repair of claim 14, wherein the first tissue is soft tissue, and the second tissue is another soft tissue.

16. The knotless locking stitch repair of claim 14, wherein the repair suture includes at least one tapered end.

17. The knotless locking stitch repair of claim 14, wherein the knotless, tensionable loop is a closed, continuous and adjustable loop having an adjustable perimeter and length.
